(19) United States
(12) Patent Application Publication
(10) Pub. No.: US 2024/0057873 A1
(43) Pub. Date: Feb. 22, 2024

(54) PHOTOACOUSTIC SIGNAL MEASUREMENT INSTRUMENT AND PHOTOACOUSTIC SIGNAL MEASUREMENT SYSTEM

(71) Applicant: FUJIFILM Healthcare Corporation, Kashiwa-shi (JP)

(72) Inventors: Rei Yokosawa, Chiba (JP); Hirozumi Takeshima, Chiba (JP); Tomohiko Tanaka, Chiba (JP)

(21) Appl. No.: 18/231,961

(22) Filed: Aug. 9, 2023

(30) Foreign Application Priority Data
Aug. 16, 2022 (JP) 2022-129698

(51) Int. Cl. A61B 5/00

(52) U.S. Cl. CPC A61B 5/0095; A61B 5/6851; A61B 5/6852

(57) ABSTRACT

A beacon device is housed within a recess portion. The beacon device has a tip end to be housed in a tip end portion. A photoacoustic wave source is disposed at the tip end of the beacon device. The tip end portion contains an ultrasonic propagation medium such as water. With the beacon device housed within the recess portion, a light generating device is connected with a rear end of the beacon device to irradiate the photoacoustic wave source with laser light. A photoacoustic signal emitted from the photoacoustic wave source is received by an ultrasonic probe. Whether the beacon device and the light generating device are connected is determined based on the received signal.

### CROSS REFERENCE TO RELATED APPLICATION

This application claims priority to Japanese Patent Application No. 2022-129698 filed on Aug. 16, 2022, which is incorporated herein by reference in its entirety including the specification, claims, drawings, and abstract.

### TECHNICAL FIELD

The present disclosure relates to a technique for measuring a photoacoustic signal from a photoacoustic wave source disposed at a tip end of a device to be inserted into a subject.

### BACKGROUND

To increase visibility of a tip end of a device inserted into a subject, techniques for visualizing the device tip with photoacoustic effect have been proposed (see JP 2019-213680 A and JP 6317847 B). Here, the device is a tubular instrument to be inserted into a blood vessel, such as a guidewire and a catheter. Hereinafter, the visualization or imaging technique using the photoacoustic effect will be referred to as a “beacon technique”, and a device with the beacon technique installed will be referred to as a “beacon device”.

An optical fiber is disposed along a beacon device. A photoacoustic wave source, such as a light absorber, is disposed at a tip end of the optical fiber to be inserted into a subject, and a light generator including a light source is disposed at a rear end opposite the tip end of the optical fiber. Light, such as laser light, emanated from the light generator irradiates, through the optical fiber, the light absorber disposed at the tip end of the optical fiber. This causes the light absorber to emit a photoacoustic signal or ultrasonic wave. The photoacoustic signal is then received by an ultrasonic imaging apparatus, which generates an image based on the received photoacoustic signal.

There have been no techniques that enable determination as to whether or not the beacon device and the light generator are properly connected to enable a photoacoustic signal source disposed at the tip end of the beacon device to properly generate photoacoustic signals, before the beacon device is actually inserted into a subject and used. It is not possible to determine, with a Sync signal, for example, the properly connected state between the light generator and the beacon device that are not electrically connected with each other.

It may be possible to manually bring the tip end of a beacon device into contact with a surface of the ultrasonic probe, prior to the actual use of the beacon device, to thereby determine proper connection between the light generator and the beacon device based on reception of a photoacoustic signal by the ultrasonic probe.

However, the beacon device that is to be inserted into a subject must be kept clean. Contact of the beacon device tip with the ultrasonic probe surface before actual use of the beacon device may compromise cleanliness of the beacon device. It is also difficult to determine proper detection of a photoacoustic signal based on an image due to multiple reflection or electrical noise generated in very shallow regions.

The present disclosure is therefore aimed toward providing a technique for determining connection between a device with a photoacoustic wave source disposed at a tip end of the device, which is to be inserted into a subject, and a light generator configured to emit light onto the photoacoustic wave source via the device.

### SUMMARY

In accordance with an aspect of the disclosure, a photoacoustic signal measurement instrument for use in measurement of a photoacoustic signal from a photoacoustic wave source disposed at a tip end of a device to be inserted into a subject includes a holder portion configured to house the tip end of the device and holding a substance that allows a photoacoustic signal to pass through, and a fixing portion configured to fix a relation between a location where an ultrasonic probe is disposed and a location of the tip end of the device, without contact between the ultrasonic probe that receives a photoacoustic signal and the tip end of the device.

This configuration enables reception of a photoacoustic signal emitted from the photoacoustic wave source by the ultrasonic probe with the tip end of the device being housed within the holder portion. This enables reception of a photoacoustic signal by the ultrasonic probe with the tip end of the device not in contact with the tip end or a transmission and reception face of the ultrasonic probe, thereby keeping the device tip clean. The holder portion holds a substance that allows a photoacoustic signal to pass through, and the photoacoustic signal is received, through the substance, by the ultrasonic probe. This reduces effects of multiple reflection and electrical noise, for example.

The photoacoustic signal measurement instrument may further include a container portion configured to house the device. The holder portion may be a recess portion on a surface of the container portion. The tip end of the device that is housed within the container portion may be contained in the recess portion. The container portion may include a space outside the recess portion to receive the ultrasonic probe facing the recess portion.

The recess portion may have a bottom face including a slope face.

The holder portion may be a container containing the substance. The fixing portion may be a through hole disposed on a surface of the container and communicating with an interior of the container, and the through hole may be configured to allow the tip end of the device that is inserted to extend through. The container may include an acoustic window configured to receive the ultrasonic probe outside the container, and the acoustic window may allow a photoacoustic signal from the photoacoustic wave source to pass through.

In accordance with another aspect of the disclosure, a photoacoustic signal measurement system includes a photoacoustic signal measurement instrument for use in measurement of a photoacoustic signal from a photoacoustic wave source disposed at a tip end of a device to be inserted into a subject, and a determination unit. The photoacoustic signal measurement instrument includes a holder portion configured to house the tip end of the device and holding a substance that allows s photoacoustic signal to pass through, and a fixing portion configured to fix a relation between a location where an ultrasonic probe is disposed and a location of the tip end of the device, without the ultrasonic probe that receives a photoacoustic signal contacting the tip end of the device. The photoacoustic wave source is irradiated with light from a light generating device via the device to thereby emit a photoacoustic signal. The determination unit is configured to determine connection between the light generating device and the device based on a photoacoustic signal emitted from the photoacoustic wave source and received by the ultrasonic probe.

The photoacoustic signal measurement system described above may further include a controller configured to control emission of light from the light generating device to the photoacoustic wave source and reception of a photoacoustic signal from the photoacoustic wave source. The controller may be configured to control execution of first processing including emission of light and reception of a photoacoustic signal in response to the emission, and second processing including reception of a photoacoustic signal with no light being emitted.

The photoacoustic signal measurement system may further include a signal processor configured to process a photoacoustic signal received by the ultrasonic probe. The signal processor may be configured to calculate a difference between a photoacoustic signal that is received by execution of the first processing and a photoacoustic signal that is received by execution of the second processing. The determination unit may be configured to determine a connection state between the light generating device and the device based on the difference.

The determination unit may be configured to determine a connection state between the light generating device and the device based on a photoacoustic signal from a region corresponding to the photoacoustic signal measurement instrument.

The region corresponding to the photoacoustic signal measurement instrument may be a region corresponding to a location of the tip end of the device fixed by the fixing portion.

The determination unit may be configured to determine a connection state between the light generating device and the device based on a ratio of a photoacoustic signal from the region corresponding to the photoacoustic signal measurement instrument and a photoacoustic signal from other regions.

The disclosure enables determination of connection between a device with a photoacoustic wave source disposed at a tip end of the device, which is to be inserted into a subject, and a light generator configured to emit light onto the photoacoustic wave source via the device.

### BRIEF DESCRIPTION OF DRAWINGS

Embodiments of the present disclosure will be described based on the following figures, wherein:

FIG. 1 is a block diagram illustrating a configuration of a photoacoustic signal measurement system according to an embodiment;

FIG. 2 is a flowchart of a process flow for measuring a photoacoustic signal in a non-contact state;

FIG. 3 is a top view of a photoacoustic signal measurement instrument as viewed from above or along the Z-axis direction;

FIG. 4 is a cross sectional view taken along A-A line in FIG. 3;

FIG. 5 is a top view of a photoacoustic signal measurement instrument as viewed from above;

FIG. 6 is a side view of a photoacoustic signal measurement instrument as viewed from a side or along the Y-axis direction;

FIG. 7 is a side view of a photoacoustic signal measurement instrument as viewed from a side or along the X-axis direction;

FIG. 8 is top view of a tip end portion of a holder portion as viewed from above;

FIG. 9 is a side view of a photoacoustic signal measurement instrument as viewed from a side or along the Y-axis direction;

FIG. 10 is a side view of a photoacoustic signal measurement instrument as viewed from a side or along the Y-axis direction;

FIG. 11 is a top view of a photoacoustic signal measurement instrument as viewed from above or along the Z-axis direction;

FIG. 12 is a top view of a photoacoustic signal measurement instrument as viewed from above or along the Z-axis direction;

FIG. 13 is a cross sectional view taken along line B-B in FIG. 12;

FIG. 14 is a side view of a photoacoustic signal measurement instrument as viewed from a side or along the Y-axis direction;

FIG. 15 is a side view of a photoacoustic signal measurement instrument as viewed from a side or along the X-axis direction;

FIG. 16 is a cross sectional view taken along line B-B in FIG. 12;

FIG. 17 is a chart indicating laser light emission timing and photoacoustic signal reception timing;

FIG. 18 is a timing chart according to related art;

FIG. 19 is a graph showing a relation among the accumulated number of frames, S/N ratio, and threshold value;

FIG. 20 illustrates a screen;

FIG. 21 shows a display example of a determination result;

FIG. 22 shows a display example of a determination result;

FIG. 23 shows a display example of a determination result;

FIG. 24 shows a display example of a determination result;

FIG. 25 shows a display example of a determination result; and

FIG. 26 illustrates a screen.

### DESCRIPTION OF EMBODIMENTS

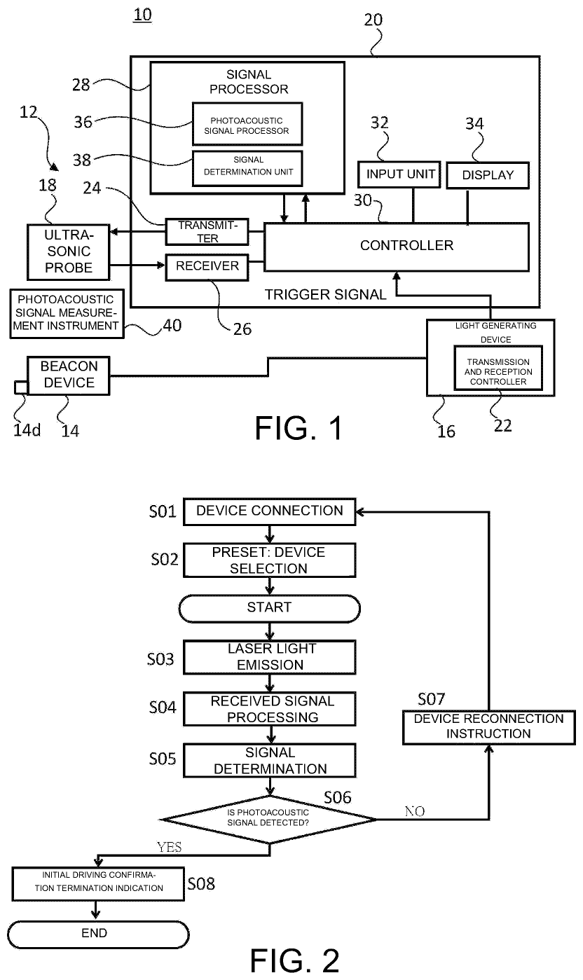

By reference to FIG. 1, a photoacoustic signal measurement system according to an embodiment will be described. FIG. 1 illustrates a configuration of a photoacoustic signal measurement system 10 according to an embodiment, which is an example system configured for assisting treatments using a beacon device.

The photoacoustic signal measurement system 10, for example, includes an ultrasonic imaging device 12, a beacon device 14, and a light generating device 16.

The ultrasonic imaging device 12 includes an ultrasonic probe 18 and a device body 20, and generates image data by transmission and reception of ultrasonic waves with the ultrasonic probe 18. The ultrasonic imaging device 12, for example, transmits an ultrasonic wave into a subject and receives an ultrasonic wave reflected within the subject to thereby generate image data representing an internal tissue of the subject.

A photoacoustic wave source 14d, such as a light absorber, is disposed at a tip end of the beacon device 14. The ultrasonic imaging device 12 receives photoacoustic signals generated by the photoacoustic wave source 14d to thereby generate image data.

The beacon device 14 is to be inserted into a subject. The beacon technique is installed in the beacon device 14. The beacon device 14 is a tubular instrument to be inserted into a blood vessel, such as a guide wire or catheter including, for example, a balloon catheter, a micro catheter, or a nutrition catheter. These examples are only illustrative, and treatments assisted by the photoacoustic signal measurement system 10 are not limited to treatments with the use of a guidewire or a catheter, and may include any treatments with the use of an instrument to be inserted into a subject.

The beacon device 14 includes an optical fiber that is not shown. The optical fiber is disposed along the beacon device 14. The photoacoustic wave source 14d is disposed at a tip end or an end of the optical fiber to be inserted into a subject, and the light generating device 16 such as a light source is disposed at a rear end of the optical fiber opposite the tip end where the photoacoustic wave source 14d is disposed.

The photoacoustic wave source 14d generates photoacoustic signals or ultrasonic waves by photoacoustic effect, for example; however, ultrasonic waves may be generated according to any principles other than the photoacoustic effect. For example, in place of the photoacoustic wave source 14d, an ultrasonic transducer such as a piezoelectric element may be disposed at the tip end of the beacon device 14 to generate ultrasonic waves. A plurality of photoacoustic wave sources 14d may be disposed, and the photoacoustic wave source 14d may be disposed at a location other than the tip end.

The light generating device 16 includes a light source that emits laser light and a transmission and reception controller 22, for example, and irradiates the photoacoustic wave source 14d with laser light to cause the photoacoustic wave source 14d to generate a photoacoustic signal.

The transmission and reception controller 22 controls the light source to thereby control emission of laser light from the light source. For example, the transmission and reception controller 22 controls emission timing of laser light. The transmission and reception controller 22 further transmits a trigger signal to receive a photoacoustic signal to the device body 20 to control the reception timing of a photoacoustic signal from the photoacoustic wave source 14d. As described above, the transmission and reception controller 22 controls emission of laser light and reception of a photoacoustic signal.

The optical fiber included in the beacon device 14 functions as a light guide member and guides the laser light emitted from the light source to the photoacoustic wave source 14d disposed at the tip end of the optical fiber. In other words, the laser light emitted from the light source passes through the optical fiber and irradiates the photoacoustic wave source 14d disposed at the tip end of the optical fiber. This causes the photoacoustic wave source 14d to generate a photoacoustic signal or an ultrasonic wave. The photoacoustic signal thus generated is received by the ultrasonic imaging device 12, which then generates image data based on the received photoacoustic signal.

The photoacoustic wave source 14d, upon receiving laser light from the light source, generates photoacoustic waves. The photoacoustic wave source 14d may include a known dye such as a photosensitizer, metal nanoparticles, or carbon-based compounds, for example. The tip end of the optical fiber and the photoacoustic wave source 14d disposed at the tip end of the optical fiber tip are covered with a resin sealing member, for example.

A photoacoustic wave emitted from the photoacoustic wave source 14d is received by the ultrasonic probe 18 disposed on a body surface of the subject. The device body 20, based on the photoacoustic wave received by the ultrasonic probe 18, generates image data representing the photoacoustic wave source 14d. The ultrasonic probe 18 further transmits an ultrasonic wave into the subject and receives an ultrasonic wave reflected within the subject. The device body 20, based on the received ultrasonic wave, generates image data, such as sectional image data, representing a tissue within the subject, such as a tissue including a blood vessel. The image representing the photoacoustic wave source 14d is superimposed on the sectional image representing the tissue within the subject, and these images are displayed on the display. This enables the operator to recognize the position of the tip end of the beacon device 14 within the subject, such as within a blood vessel.

The ultrasonic probe 18 transmits and receives ultrasonic waves, and includes a 1D array transducer including a plurality of ultrasonic transducers arranged one-dimensionally. The 1D array transducer forms an ultrasonic beam, which is electronically scanned in a repeating manner to thereby form a scan plane corresponding to a two-dimensional echo data capturing space, within a living body for each electronic scanning. The ultrasonic probe 18 may include a 2D array transducer including a plurality of transducers arranged two-dimensionally, in place of the 1D array transducer. The 2D array transducer forms an ultrasonic beam, which is electronically scanned in a repeating manner to thereby form a scan plane or a two-dimensional echo data capturing space for each electronic scan. Scanning of the ultrasonic beam two-dimensionally forms a three-dimensional scan plane or a three-dimensional echo data capturing space. Scanning methods include sector scanning, linear scanning, and convex scanning, for example. The ultrasonic probe 18 disposed on the body surface may be replaced with an ultrasonic probe disposed at a location other than on the body surface, such as an ultrasonic probe for use in intravascular ultrasound (IVUS).

The device body 20 includes a transmitter 24, a receiver 26, a signal processor 28, a controller 30, an input unit 32, and a display 34.

The transmitter 24 functions as a transmitting beam former and the receiver 26 functions as a received beam former. At the time of transmission, the transmitter 24 provides a plurality of transmitting signals having a predetermined delay relation to the ultrasonic transducers included in the ultrasonic probe 18, thereby forming an ultrasonic transmitting beam. At the time of reception, the ultrasonic probe 18 receives a reflection wave (RF signal) from within the living body and outputs a plurality of received signals to the receiver 26. The receiver 26 applies phase alignment and summing processing to the received signals to thereby form a received beam, and the beam data are then output to the signal processor 28. More specifically, the receiver 26 applies delay processing to a received signal acquired from each ultrasonic transducer according to a delay processing condition of each ultrasonic transducer, and performs summing processing of a plurality of received signals acquired from the ultrasonic transducers, thereby forming the received beam. The delay processing condition is defined by reception delay data indicating delay time. A set of reception delay data or a set of delay time corresponding to the plurality of ultrasonic transducers is supplied from the controller 30.

With the operation of the transmitter 24 and the receiver 26, the ultrasonic beams; that is, the transmitting beams and the received beams, are electronically scanned to form a scan plane corresponding to a plurality of sets of beam data that form received frame data, and more specifically, RF signal frame data. Each set of beam data is composed of a plurality of echo data items arranged along the depth direction. After repeating electronic scanning of the ultrasonic beam, a plurality of sets of reception frame data arranged on the time axis, which form a reception frame sequence, are output by the receiver 26.

Electronic scanning of the ultrasonic beam two-dimensionally with the operation of the transmitter 24 and the receiver 26 forms a three-dimensional echo data capturing space, from which volume data or a set of echo data is acquired. Repeating the electronic scanning of the ultrasonic beam results in output of the plurality of volume data arranged on the time axis from the receiver 26, which form a volume data sequence.

The signal processor 28 applies, to the beam data output from the receiver 26, signal processing including detection, logarithmic compression, and a conversion function such as a coordinate transformation function and interpolation processing function by a digital scan converter (DSC), for example, to generate image data (e.g., B mode image).

The controller 30 controls transmission and reception of ultrasonic waves by the ultrasonic probe 18, and generates image data based on the ultrasonic waves received by the ultrasonic probe 18. The controller 30 further controls the timing and number of image capturing of reflected ultrasonic images and the timing and number of image capturing of photoacoustic images.

The reflected ultrasonic image is image data generated based on the ultrasonic waves transmitted from the ultrasonic probe 18 and reflected within the subject, or reflected waves. More specifically, the reflected ultrasonic image is image data generated based on reflected ultrasonic signals output from the receiver 26 that has received the reflected ultrasonic waves. Imaging of a reflected ultrasonic image refers to transmitting ultrasonic waves from the ultrasonic probe 18 and receiving the reflected waves of the transmitted ultrasonic waves, thereby generating a reflected ultrasonic image. In the following description, imaging of the reflected ultrasonic image will be referred to as “reflected ultrasonic imaging”.

The photoacoustic image is image data generated based on photoacoustic waves from the photoacoustic wave source 14d. More specifically, the photoacoustic image is image data generated based on photoacoustic signals output from the receiver 26 that has received the photoacoustic waves. Imaging of a photoacoustic image refers to generating a photoacoustic image based on photoacoustic waves from the photoacoustic wave source 14d. In the following description, imaging of a photoacoustic image will be referred to as “photoacoustic imaging”.

The controller 30 controls the number of imaging, imaging period, and imaging timing of each of reflected ultrasonic imaging and photoacoustic imaging. To perform reflected ultrasonic imaging, the controller 30 controls transmission of ultrasonic waves by the transmitter 24 and reception of ultrasonic waves by the receiver 26. This control results in generation of reflected ultrasonic signals, and based on the reflected ultrasonic signals, a reflected ultrasonic image is generated. The controller 30 further receives a trigger signal for reception of a photoacoustic signal from the light generating device 16, to control reception of photoacoustic waves by the receiver 26 in accordance with the trigger signal. This results in generation of a photoacoustic signal, which is used to generate a photoacoustic image. The controller 30 may include the transmission and reception controller 22 to control the timing for emitting laser light to the photoacoustic wave source 14d and the timing for receiving photoacoustic waves by the receiver 26.

The signal processor 28 includes a photoacoustic signal processor 36 and a signal determination unit 38.

The photoacoustic signal processor 36 performs photoacoustic imaging to apply signal processing to a photoacoustic signal output from the receiver 26, thereby generating a photoacoustic image. More specifically, the photoacoustic signal processor 36 images a photoacoustic signal to thereby generate a photoacoustic image.

The controller 30, for example, generates a display image by superimposing a photoacoustic image on a reflected ultrasonic image, and displays the display image on the display 34.

The signal determination unit 38 is an example determination unit and determines a connection state between the light generating device 16 and the beacon device 14 based on a photoacoustic signal emitted by the photoacoustic wave source 14d and received by the ultrasonic probe 18, The controller 30 causes the display 34 to display information indicating the determination result.

The transmission and reception controller 22 controls execution of first processing and execution of second processing, for example. The first processing includes transmission or emission of laser light and reception of a photoacoustic signal in response to the transmission. The second processing includes reception of a photoacoustic signal with no laser light being transmitted.

The photoacoustic signal processor 36 calculates a difference between a photoacoustic signal received through execution of the first processing and a photoacoustic signal received through execution of the second processing. The signal determination unit 38, based on the difference, determines the connection state between the light generating device 16 and the beacon device 14.

The input unit 32 is a device to enable a user to input, to the device body 20, conditions and instructions that are necessary for imaging, for example, and may be an operation panel, a switch, a button, a keyboard, a mouse, or a joystick, for example.

The display 34 is a liquid crystal display or an EL display, for example. The display 34 displays a reflected ultrasonic image and a photoacoustic image. The display 34 may further display a reflected ultrasonic image and a photoacoustic image with the photoacoustic image superimposed on the reflected ultrasonic image. The display 34 may include both a display and the input unit 32, and may implement a Graphic User Interface (GUI), for example. The display 34 may further implement a user interface such as a touch panel.

The device body 20 includes a storage device that is not shown. The storage device includes one or more storage regions for storing data, and may be a hard disk drive (HDD), a solid-state drive (SSD), various memories, such as RAM, DRAM, and ROM, or other storage devices such as an optical disk, or combinations of these devices. A reflected ultrasonic image and a photoacoustic image, for example, are stored in the storage device.

Each of the transmission and reception controller 22, the signal processor 28, and the controller 30 may be implemented with a hardware resource such as a processor or an electronic circuit, for example, and a device such as a memory may be employed as required in the implementation. Each of the transmission and reception controller 22, the signal processor 28, and the controller 30 may also be implemented with a computer.

More specifically, each of the transmission and reception controller 22, the signal processor 28, and the controller 30 may be entirely or partially implemented with cooperation of a hardware resource such as a Central Processing Unit (CPU) and a memory included in a computer and a software or a program that defines operation of the CPU and the like. Such a program is stored in the storage device through a storage medium such as a CD and a DVD, or via a communication network, for example. Alternatively, each of the transmission and reception controller 22, the signal processor 28, and the controller 30 may be implemented by a Digital Signal Processor (DSP), an Application Specific Integrated Circuit (ASIC), or a Field Programmable Gate Array (FPGA), for example. A Graphics Processing Unit (GPU), for example, may also be employed. Each of the transmission and reception controller 22, the signal processor 28, and the controller 30 may be implemented by a single device, or each function may be implemented by one or more devices.

The photoacoustic signal measurement system 10 further includes a photoacoustic signal measurement instrument 40 that houses the beacon device 14. The photoacoustic signal measurement instrument 40 is further configured to measure a photoacoustic signal emitted from the photoacoustic wave source 14d, with the ultrasonic probe 18 and the beacon device 14 not in contact with each other. For example, prior to insertion of the beacon device 14 into the subject for actual use, a photoacoustic signal is measured with the beacon device 14 being housed within the photoacoustic signal measurement instrument 40. The signal determination unit 38, based on the measured photoacoustic signal, determines the connection state between the light generating device 16 and the beacon device 14. The controller 30 causes the display 34 to display information indicating the determination result. The specific configuration of the photoacoustic signal measurement instrument 40 will be described in detail below.

By reference to FIG. 2, a process flow for measuring a photoacoustic signal, with the ultrasonic probe 18 and the beacon device 14 not in contact with each other, will be described below. FIG. 2 shows a flowchart indicating a process flow for measuring a photoacoustic signal in a non-contact state.

First, the beacon device 14 housed in the photoacoustic signal measurement instrument 40 is connected to the light generating device 16 (S01). In this state, the photoacoustic wave source 14d is housed within the photoacoustic signal measurement device 40. In some examples, the ultrasonic imaging device 12 may select the type of the beacon device 14, as a preset (S02).

The light generating device 16 then irradiates the photoacoustic wave source 14d with laser light (S03). Specifically, the light generating device 16 irradiates the photoacoustic wave source 14d via the optical fiber included in the beacon device 14, which causes the photoacoustic wave source 14d to generate a photoacoustic signal.

The photoacoustic signal emitted from the photoacoustic wave source 14d is received by the ultrasonic probe 18 that is disposed on a surface of the photoacoustic signal measurement instrument 40. Specifically, the ultrasonic probe 18 receives a photoacoustic signal emitted from the photoacoustic wave source 14d of the beacon device 14 housed in the photoacoustic signal measurement instrument 40.

The photoacoustic signal received by the ultrasonic probe 18 is processed by the receiver 26 and the photoacoustic signal processor 36 (S04).

The signal determination unit 38 determines, based on the photoacoustic signal, whether or not the beacon device 14 and the light generating device 16 are connected (S05). Specifically, the signal determination unit 38 determines whether the ultrasonic probe 18 receives and detects a photoacoustic signal.

In response to detection of no photoacoustic signal (NO in step S06), the controller 30 outputs information indicating an instruction to reconnect the beacon device 14 (step S07). For example, the controller 30 causes the display 34 to display a message or the like that encourages a user such as an operator to check the connection between the beacon device 14 and the light generating device 16. Thereafter, processing in each of step S01 and the following steps is performed.

In response to detection of a photoacoustic signal (YES in step S06), the controller outputs information indicating completion of check of the initial driving of the beacon device 14 (step S08). For example, the controller 30 causes the display 34 to display a message and the like indicating completion of check of the initial driving.

This configuration enables the user such as an operator to confirm a state of connection between the beacon device 14 and the light generating device 16, with the beacon device 14 being housed within the photoacoustic signal measurement instrument 40.

### Photoacoustic Signal Measurement Instrument

The photoacoustic signal measurement instrument 40 will be described below. The photoacoustic signal measurement instrument 40 includes a holder portion that stores a tip of the beacon device 14 and also contains a substance that allows photoacoustic signals to pass through. The photoacoustic signal measurement instrument 40 further includes a fixing portion that fixes a relation between a location where the ultrasonic probe 18 is disposed and a location of the tip end of the beacon device 14 without bringing the ultrasonic probe 18 into contact with the tip end of the beacon device 14. Specific examples of the photoacoustic signal measurement instrument 40 will be described below.

### Example 1

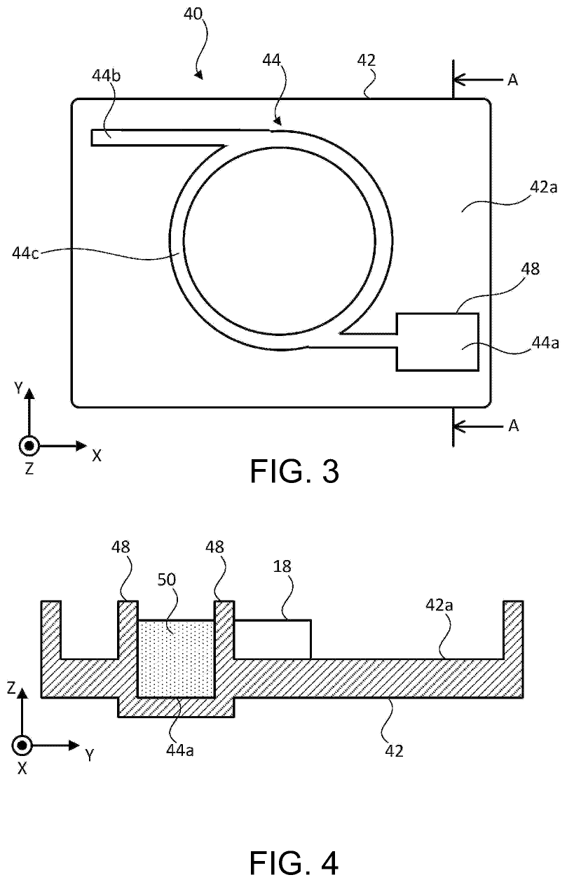

By reference to FIG. 3 to FIG. 8, the photoacoustic signal measurement instrument 40 or a photoacoustic signal measurement instrument according to EXAMPLE 1 will be described. Here, a three-dimensional orthogonal coordinates system is defined; an X-axis, a Y-axis, and a Z-axis are mutually orthogonal.

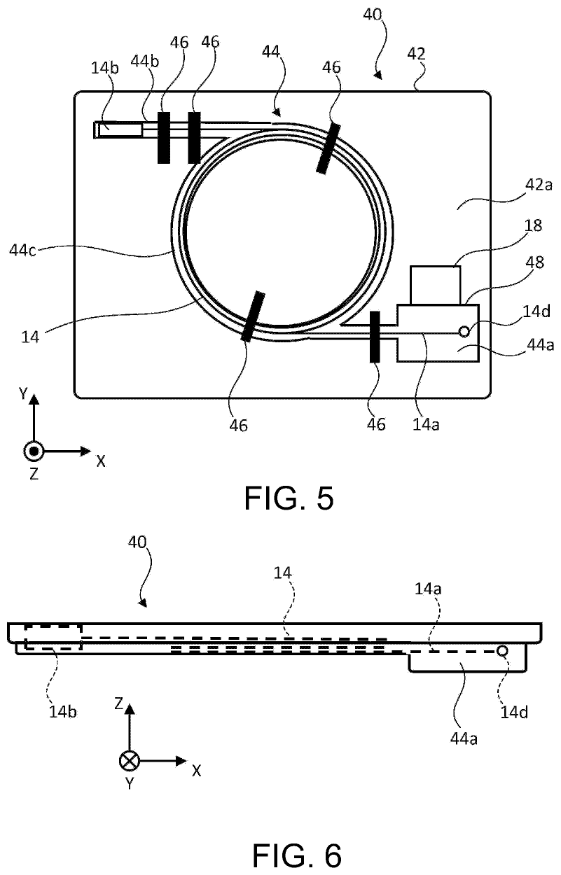

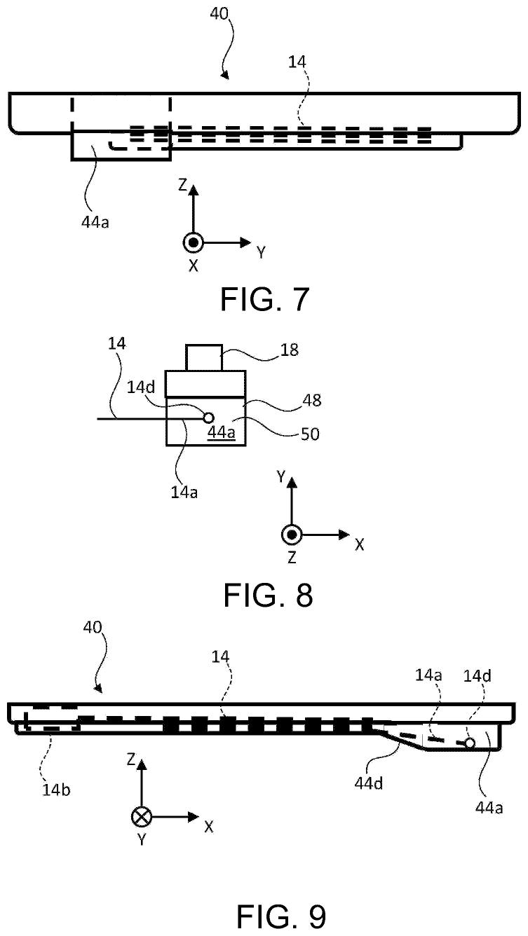

FIG. 3 is a top view of the photoacoustic signal measurement instrument 40 as viewed from above (along the Z-axis). FIG. 4 is a cross sectional view taken along line A-A in FIG. 3. FIG. 3 and FIG. 4 illustrate the photoacoustic signal measurement instrument with the beacon device 14 not being housed. FIG. 5 is a top view of the photoacoustic signal measurement instrument 40 as viewed from above. FIG. 6 is a side view of the photoacoustic signal measurement instrument 40 as viewed from a side along the Y-axis. FIG. 7 is a side view of the photoacoustic signal measurement instrument 40 as viewed from a side along the X-axis. FIG. 8 is a top view of a tip end portion of the holder portion as viewed from above.

The photoacoustic signal measurement instrument 40 includes a packing container 42, a recess portion 44, and an adhesion tape 46. The packing container 42 is a sterilized container for packing, for example, and may be a thin container made of resin such as polypropylene.

The recess portion 44 is an example of the holder portion and is disposed in one face 42a of the packing container 42. The recess portion 44 is a channel, for example. The beacon device 14 is housed and retained within the recess portion 44. For example, a wall 48 forms the recess portion 44. Specifically, the wall 48 is disposed on the face 42a to form the recess portion 44.

The recess portion 44 includes a tip end portion 44a, a rear end portion 44b, and a middle portion 44c. The tip end portion 44a is a recess located at the tip end of the recess portion 44. The rear end portion 44b is a recess located at the rear end of the recess portion 44. The middle portion 44c is a recess located between the tip end portion 44a and the rear end portion 44b to connect the tip end portion 44a and the rear end portion 44b.

The tip end portion 44a houses the tip end 14a of the beacon device 14, and the rear end portion 44b houses a rear end 14b of the beacon device 14. The middle portion 44c houses a middle portion, portions other than the tip end 14a and the rear end 14b, of the beacon device 14. The middle portion 44c is annular when viewed from above or along the Z-axis.

As illustrated in FIG. 5 to FIG. 7, the beacon device 14 is wound to fit the annular middle portion 44c and is housed within the recess portion 44. At this time, the tip end 14a of the beacon device 14 is housed within the tip end portion 44a of the recess portion 44 and the rear end 14b of the beacon device 14 is housed within the rear end portion 44b of the recess portion 44; however, this is one example housing manner, and the beacon device 14 may be folded in any shape other than an annular shape and then be housed within the recess portion 44.

The adhesion tape 46 fixes the beacon device 14 housed within the recess portion 44. As illustrated in FIG. 5, a plurality of adhesion tapes 46 are attached to cross over the beacon device 14 housed within the recess portion 44. This enables the beacon device 14 housed within the recess portion 44 to be fixed to the packing container 42.

As illustrated in FIG. 5, the packing container 42 further includes a space outside the recess portion 44, where the ultrasonic probe 18 is disposed facing the recess portion 44. More specifically, a space on the face 42a outside the tip end portion 44a is used for placing the ultrasonic probe 18. For example, the tip end, or a transmission and reception face, of the ultrasonic probe 18 comes into contact with the wall 48 forming the tip end portion 44a. The tip end 14a of the beacon device 14 is housed within the tip end portion 44a of the recess portion 44, and the inside and outside of the tip end portion 44a are separated via the wall 48; the transmission and reception face of the ultrasonic probe 18 does not therefore contact the tip end 14a of the beacon device 14. As described above, housing the beacon device 14 within the recess portion 44 enables fixing of the relation between the location where the ultrasonic probe 18 is disposed and the location of the tip end 14a of the beacon device 14, with the tip end 14a of the beacon device 14 not in contact with the ultrasonic probe 18. In EXAMPLE 1, the recess portion 44 functions as an example of the fixing portion. As the adhesion tape 46 firmly fixes the beacon device 14, the recess portion 44 and the adhesion tape 46 may function as an example of the fixing portion.

The photoacoustic wave source 14d is disposed at the tip end 14a of the beacon device 14. To measure photoacoustic signals emitted from the photoacoustic wave source 14d, a transmission substance 50 is contained within the recess portion 44, as illustrated in FIG. 4 and FIG. 8. The transmission substance 50 allows the photoacoustic signal to pass through, and is sterilized water, normal saline, or ultrasonic jelly, for example.

With the beacon device 14 and the transmission substance 50 being contained within the recess portion 44, the ultrasonic probe 18 is disposed outside the tip end portion 44a of the recess portion 44, as illustrated in FIG. 5 and FIG. 8. At this time, the ultrasonic probe 18 is disposed such that the tip end of the ultrasonic probe 18 is in contact with the wall 48 that forms the tip end portion 44a of the recess portion 44.

The rear end 14b of the beacon device 14 is connected with the light generating device 16. For example, the rear end 14b is removed from the recess portion 44 and is connected with the light generating device 16. Laser light emitted from the light generating device 16 passes through the optical fiber in the beacon device 14 and irradiates the photoacoustic wave source 14d disposed at the tip end 14a of the beacon device 14. This results in generation of a photoacoustic signal from the photoacoustic wave source 14d, and the photoacoustic signal is then received by the ultrasonic probe 18 disposed outside the tip end portion 44a of the recess portion 44. The signal determination unit 38, based on a photoacoustic signal received by the ultrasonic probe 18, determines whether the beacon device 14 and the light generating device 16 are connected. More specifically, the signal determination unit 38 determines whether the rear end 14b of the beacon device 14 and the light generating device 16 are connected and the ultrasonic probe 18 detects a photoacoustic signal.

As described above, the photoacoustic signal measurement instrument 40 according to EXAMPLE 1 enables determination of whether the beacon device 14 is connected with the light generating device 16, with the beacon device 14 being housed within the recess portion 44. The beacon device 14 housed within the recess portion 44 enables determination of whether the beacon device 14 is connected to the light generating device 16 without making the tip end or transmission and reception face of the ultrasonic probe 18 in contact with the tip end 14a of the beacon device 14. This enables determination of whether the beacon device 14 is connected with the light generating device 16 while keeping the beacon device 14 clean.

Further, the ultrasonic probe 18 receives a photoacoustic signal through the transmission substance 50 contained within the recess portion 44. This configuration reduces generation of multiple reflections or generation of electric noises in very shallow regions, for example.

FIG. 9 illustrates Modification Example 1 of EXAMPLE 1. FIG. 9 is a side view of the photoacoustic signal measurement instrument 40 as viewed from a side along the Y-axis direction.

As illustrated in FIG. 9, a bottom face 44d of the tip end portion 44a may include a slope face. In this example, the bottom face 44d of the tip end portion 44a includes a slope face that is sloped downward from a boundary between the tip end portion 44a and the middle portion 44c toward the tip end portion 44a such that tip end portion 44a has an increased depth. This allows the tip end 14a of the beacon device 14 to be inclined within the tip end portion 44a. The transmission substance 50 is contained within the slope portion where the photoacoustic wave source 14d emits a photoacoustic signal, which is received by the ultrasonic probe 18.

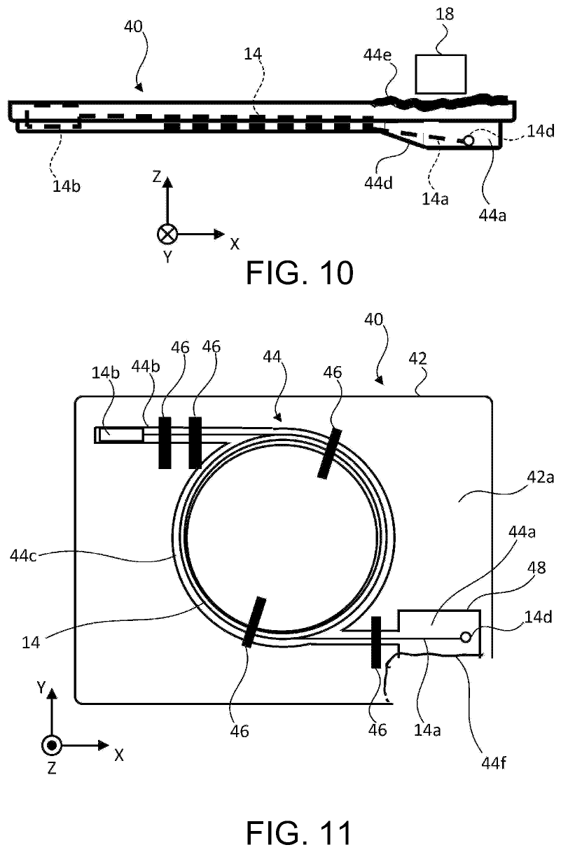

FIG. 10 illustrates Modification Example 2 of EXAMPLE 1. FIG. 10 is a side view of the photoacoustic signal measurement instrument 40 as viewed from a side face along the Y-axis.

As illustrated in FIG. 10, a sheet 44e is disposed at the top of the tip end portion 44a. The sheet 44e is a member that allows ultrasonic waves to pass through and may be a film, for example. The transmission and reception face of the ultrasonic probe 18 is brought into contact with the sheet 44e to enable the ultrasonic probe 18 to receive a photoacoustic signal emitted from the photoacoustic wave source 14d. The ultrasonic probe 18 may be disposed as illustrated in FIG. 8. The sheet 44e may be preset on the tip end portion 44a or may be placed by a user such as an operator at the time of measurement of a photoacoustic signal. The bottom face 44d of the tip end portion 44a may or may not include a slope face.

FIG. 11 illustrates Modification Example 3 of EXAMPLE 1. FIG. 11 is a top view of the photoacoustic signal measurement instrument 40 as viewed from above along the Z-axis.

As illustrated in FIG. 11, a side face of the tip end portion 44a is cut out to form a cut portion 44f for measuring photoacoustic signals. As a wall similar to the wall 48 is disposed in the cut portion, the tip end portion 44a, which includes a cut side face, contains an ultrasonic wave propagation medium such as water. To enable formation of the cut portion 44f, the tip end portion 44a includes, on the side face, folds and perforations. The transmission and reception face of the ultrasonic probe 18 is brought into contact with the cut portion 44f to enable the ultrasonic probe 18 to receive a photoacoustic signal emitted from the photoacoustic wave source 14d. The ultrasonic probe 18 may be disposed as illustrated in FIG. 8. The bottom face 44d of the tip end portion 44a may or may not include a slope face.

### Example 2

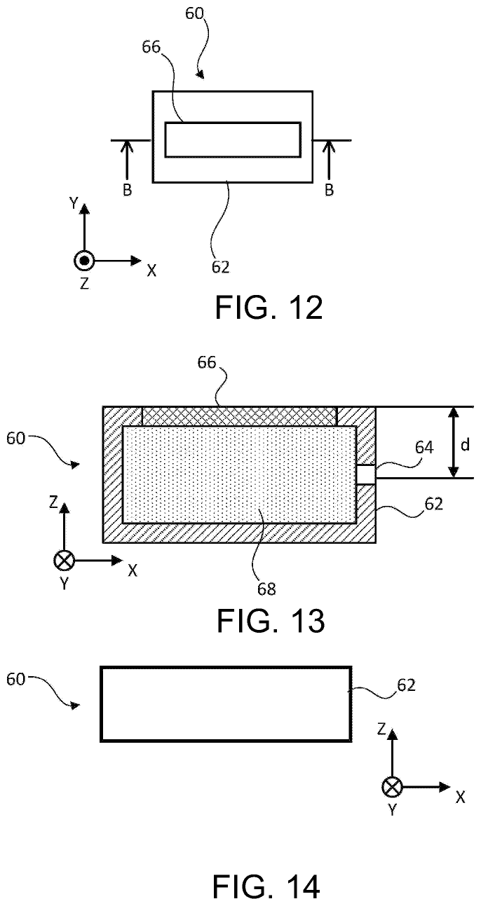

By reference to FIG. 12 to FIG. 16, a photoacoustic signal measurement instrument 60 that is a photoacoustic signal measurement instrument according to EXAMPLE 2 will be described.

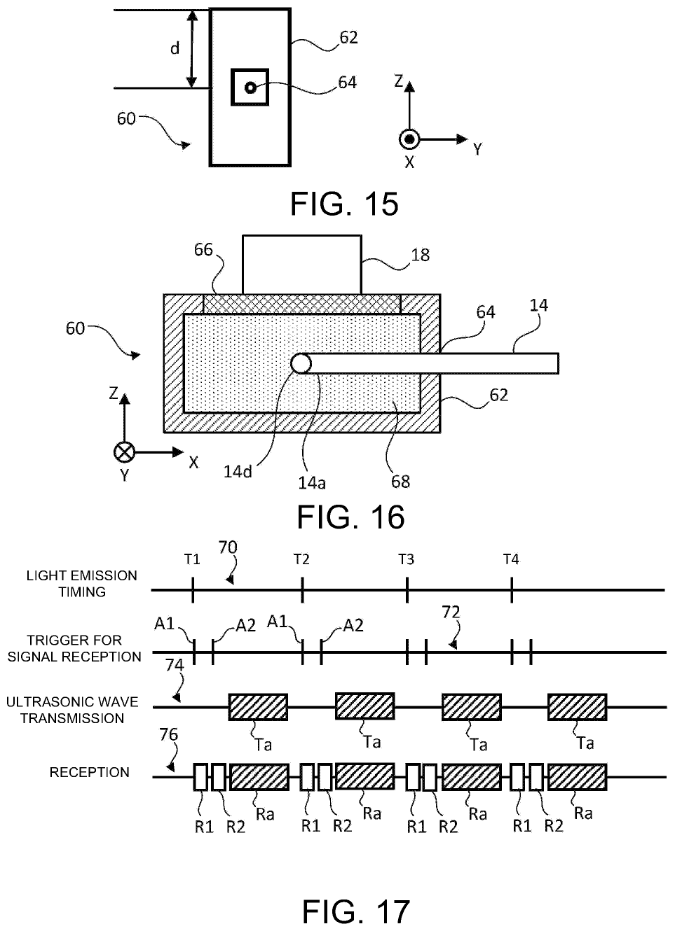

FIG. 12 is a top view of the photoacoustic signal measurement instrument 60 as viewed from above along the Z-axis. FIG. 13 is a cross sectional view taken along B-B line in FIG. 12, and illustrates a cross section of the photoacoustic signal measurement instrument 60 without the tip end 14a of the beacon device 14 inserted in the photoacoustic signal measurement instrument 60. FIG. 14 is a side view of the photoacoustic signal measurement instrument 60 as viewed from a side along the Y-axis. FIG. 15 is a side view of the photoacoustic signal measurement instrument 60 as viewed from a side along the X-axis. FIG. 16 is a cross sectional view taken along B-B line in FIG. 12, and illustrates a cross section of the photoacoustic signal measurement instrument 60 with the tip end 14a of the beacon device 14 being inserted in the photoacoustic signal measurement instrument 60.

The photoacoustic signal measurement instrument 60 includes a container 62 that is an example holder portion. The container 62 is a sterilized box, for example. As illustrated in FIG. 13 and FIG. 15, the container 62 includes, on a side thereof, a through hole 64 connecting to the interior of the container 62. The through hole 64 corresponds to an example of the fixing portion. The tip end 14a of the beacon device 14 is to be inserted into the through hole 64. The through hole 64 has a size that allows the beacon device 14 to extend through but does not allow formation of a gap between the beacon device 14 that is inserted and the through hole 64. The beacon device 14, upon being inserted in the through hole 64, is supported by the container 62, and the position of the beacon device 14 is fixed.

An acoustic window 66 is disposed on a top face of the container 62. The acoustic window 66 is a member that allows ultrasonic waves to pass through. As described below, the ultrasonic probe 18, when disposed on the acoustic window 66, is disposed outside the container 62. The through hole 64 is disposed on a face other than a face where the acoustic window 66 is disposed. As illustrated in FIG. 13, the container 62 is filled with a transmission substance 68 that allows a photoacoustic signal to pass through, and is sterilized water, normal saline, or ultrasonic jelly, for example. Prior to the use of the photoacoustic signal measurement instrument 60, a sealing member that closes the through hole 64 is attached to the face including the through hole 64. To use the photoacoustic signal measurement instrument 60, the sealing member is removed and the tip end 14a of the beacon device 14 is inserted into the through hole 64.

As illustrated in FIG. 16, the tip end 14a of the beacon device 14 is inserted in the through hole 64, and then inserted, through the through hole 64, into the container 62. The container 62 is filled with the transmission substance 68, and the tip end 14a of the beacon device 14 is placed within the transmission substance 68. Further, the ultrasonic probe 18 is disposed on the acoustic window 66 such that the transmission and reception face of the ultrasonic probe 18 is in contact with the acoustic window 66. The beacon device 14 inserted through the through hole 64 is supported by the container 62, and the relation between the location where the ultrasonic probe 18 is disposed or the position of the acoustic window 66, and the tip end 14a of the beacon device 14, is fixed.

Similar to EXAMPLE 1, the rear end 14b of the beacon device 14 is connected to the light generating device 16. Laser light emitted from the light generating device 16 passes through the optical fiber in the beacon device 14 and irradiates the photoacoustic wave source 14d disposed at the tip end 14a of the beacon device 14. This causes the photoacoustic wave source 14d to generate and emit a photoacoustic signal externally of the container 62 via the acoustic window 66. The photoacoustic signal is then received by the ultrasonic probe 18 disposed on the acoustic window 66.

The signal determination unit 38, based on a photoacoustic signal received by the ultrasonic probe 18, determines whether the beacon device 14 and the light generating device 16 are connected. More specifically, the signal determination unit 38 determines whether the rear end 14b of the beacon device 14 and the light generating device 16 are connected to enable the ultrasonic probe 18 to detect a photoacoustic signal.

As described above, the photoacoustic signal measurement instrument 60 according to EXAMPLE 2 enables determination as to whether the beacon device 14 is connected to the light generating device 16, with the beacon device 14 being supported by the container 62. The tip end 14a of the beacon device 14 housed within the container 62 allows determination of whether the beacon device 14 is connected with the light generating device 16, with the transmission and reception face of the ultrasonic probe 18 not in contact with the tip end 14a of the beacon device 14. This enables determination of whether the beacon device 14 is connected to the light generating device 16 while keeping the beacon device 14 clean.

The container 62 is filled with the transmission substance 68, through which a photoacoustic signal is received by the ultrasonic probe 18. This enables reduction in generation of multiple reflections and generation of electrical noise in very shallow regions, for example.

A support member that supports the ultrasonic probe 18 may be disposed on the top face of the container 62, including the acoustic window 66. For example, a support member shaped to cover the tip end of the ultrasonic probe 18, such as a frame, may be disposed on the top face to receive the tip end of the ultrasonic probe 18 fitted into the support member, thereby fixing the ultrasonic probe 18 on the top face. The relation between the location of the ultrasonic probe 18 and the location of the tip end 14a of the beacon device 14 may be fixed in this manner.

A sound-absorbing material that prevents reflection of sound may be disposed in the photoacoustic signal measurement instruments 40 and 60.

Method for Controlling Light Emission to Photoacoustic Wave Source 14d and Reception of Photoacoustic Signal

A method for controlling light emission to the photoacoustic wave source 14d and reception of a photoacoustic signal will be described below.

By reference to FIG. 17, the emission timing of laser light to the photoacoustic wave source 14d and the reception timing of a photoacoustic signal will be described.

Reference numeral 70 indicates emission timing of laser light to be emitted from the light generating device 16. Reference numeral 72 indicates generation timing of a trigger signal that instructs reception of a photoacoustic signal. The trigger signal is output from the light generating device 16 to the controller 30 in response to emission of laser light by the light generating device 16. Reference numeral 74 indicates transmission timing of an ultrasonic wave from the ultrasonic probe 18. Reference numeral 76 indicates reception timing of an ultrasonic wave by the ultrasonic probe 18.

During a period Ta, ultrasonic waves are transmitted from the ultrasonic probe 18, and during a period Ra, in response to the transmission of an ultrasonic wave, an ultrasonic wave or a reflected wave is received by the ultrasonic probe 18.

At each time points T1, T2, T3, and T4, the light generating device 16 emits laser light to the photoacoustic wave source 14d.

A trigger signal A1 is output from the light generating device 16 to the controller at each of the time points T1, T2, T3, and T4. More specifically, the trigger signal A1 is output from the light generating device 16 to the controller 30 in response to emission of laser light. A trigger signal A2 is output from the light generating device 16 to the controller 30 during a period in which laser light is not emitted by the light generating device 16. The trigger signal A2 is output from the light generating device 16 to the controller 30 after the trigger signal A1 is output, for example; however, the trigger signal A2 may be output at any time point in a period in which laser light is not emitted, such as before the trigger signal A1 is output.

During periods R1 and R2, the ultrasonic probe 18 receives photoacoustic signals. Each of the periods R1 and R2 is a period corresponding to emission of laser light from the light generating device 16 to the photoacoustic wave source 14d. The period R1 corresponds a period related to the trigger signal A1, and the period R2 corresponds to a period related to the trigger signal A2. In other words, the period R1 corresponds to a period related to a time point at which laser light is emitted, and the period R2 corresponds to a period in which laser light is not emitted.

The controller 30 controls emission of laser light from the light generating device 16 to the photoacoustic wave source 14d and reception of a photoacoustic signal from the photoacoustic wave source 14d. Specifically, the controller 30 controls the emission timing of laser light and the reception timing of a photoacoustic signal. The transmission and reception controller 22 and the controller 30 may be cooperated to control the emission timing of laser light and the reception timing of a photoacoustic signal.

The controller 30 controls first processing and second processing, for example. The first processing includes emission of laser light and corresponding reception of a photoacoustic signal. The second processing includes reception of a photoacoustic signal with no laser light being emitted.

In the example illustrated in FIG. 17, the first processing is a combination of emission of laser light at the time point T1 and reception of a photoacoustic signal during the period R1 related to the trigger signal A1. The second processing is reception of a photoacoustic signal during the period R2 related to the trigger signal A2.

At the time point T1, laser light is emitted, and in response to the emission, a photoacoustic signal is received in the period R1 related to the trigger signal A1. Subsequently, at a time point when no laser light is emitted, the trigger signal A2 is output from the light generating device 16 to the controller 30, and in response to the trigger signal A2, a photoacoustic signal is received in the period R2 related to the trigger signal A2. Hereinafter, these operations will be described in detail.

At the time point T1, the controller 30 causes the light generating device 16 to generate laser light. This causes the photoacoustic wave source 14d to be irradiated with the laser light and generate a photoacoustic signal. In response to emission of laser light from the light generating device 16 to the photoacoustic wave source 14d, the light generating device 16 outputs the trigger signal A1 related to the time point T1 to the controller 30, and also outputs to the controller 30 the trigger signal A2 related to a period in which laser light is not emitted.

The controller 30 controls reception of an ultrasonic wave or a photoacoustic signal by the ultrasonic probe 18 and the receiver 26 during the period R1 corresponding to the trigger signal A1. The controller 30 further controls reception of ultrasonic wave or a photoacoustic signal by the ultrasonic probe 18 and the receiver 26 during the period R2 corresponding to the trigger signal A2. This control enables reception of a photoacoustic signal by the ultrasonic probe 18 and the receiver 26 during the period R1 and reception of a photoacoustic signal by the ultrasonic probe 18 and the receiver 26 during the period R2.

At the time point T2 and the following time points, emission of laser light, reception during the period R1, and reception during the period R2 are similarly performed.

In the following description, a photoacoustic signal received during the period R1 will be referred to as a “photoacoustic signal S1” and a photoacoustic signal received during the period R2 as a “photoacoustic signal S2”.

The period R2 corresponds to a period in which laser light is not emitted. The photoacoustic signal S2 received during the period R2 therefore corresponds to a signal, such as noise, received by the ultrasonic probe 18 and the receiver 26 when no laser light is emitted.

In the example illustrated in FIG. 17, immediately after the first processing to perform emission of laser light and reception of a photoacoustic signal, the second processing to receive a photoacoustic signal with no laser light being emitted is performed. More specifically, transmission and reception of ultrasonic waves or transmission during the period Ta and reception during the period Ra are not performed between the first processing and the second processing, and the first processing and the second processing are performed successively. In another example, transmission and reception of ultrasonic waves may be performed between the first processing and the second processing, and the first processing and the second processing may not be performed successively. For example, the first processing, the transmission and reception of ultrasonic waves, and the second processing are sequentially performed in this order. In this case, the reception periods are defined in the order of the period R1, the period Ra, and then the period R2.

In a further example, the controller 30 may switch emission of laser light from the light generating device 16 ON and OFF for each predetermined period, or may perform predetermined code transmission.

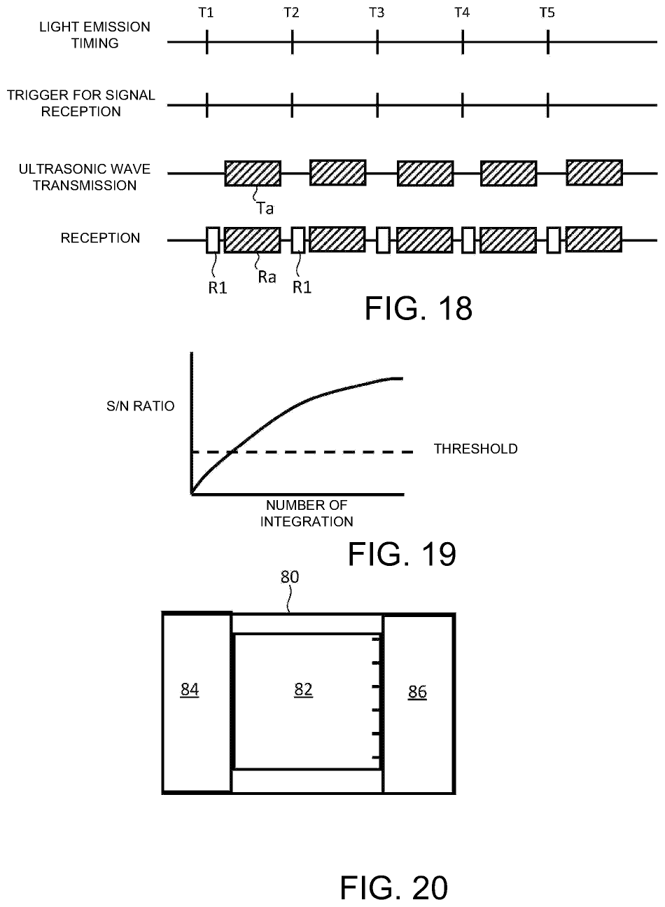

FIG. 18 shows timing, for example, according to the related art. In this example, a photoacoustic signal is received during the period R1 corresponding to emission of laser light, and the period R2 is not set.

The photoacoustic signal processor 36 calculates a difference between a photoacoustic signal received with execution of the first processing and a photoacoustic signal received with execution of the second processing. The signal determination unit 38, based on the difference, determines a connection state between the light generating device 16 and the beacon device 14.

The photoacoustic signal received with execution of the first processing corresponds to the photoacoustic signal S1, and the photoacoustic signal received with execution of the second processing corresponds to the photoacoustic signal S2.

The photoacoustic signal processor 36 calculates a difference ΔS between the photoacoustic signal S1 received during the period R1 and the photoacoustic signal S2 received during the period R2. The photoacoustic signal S2 is a photoacoustic signal received during the period R2 corresponding to the period in which laser light is not emitted. Calculation of the difference between the photoacoustic signal S1 and the photoacoustic signal S2 therefore removes a signal such as noise, thereby providing a difference ΔS corresponding to a photoacoustic signal with enhanced intensity.

The method for acquiring the difference ΔS is not limited to the above example; the difference ΔS may be acquired with the use of a signal showing an envelope, an RF signal, or image data, for example. The photoacoustic signal processor 36 may perform peak hold processing and frame integrations multiple times. The number of images for use in peak hold and frame integration is not limited, because it is not necessary to consider the effect of body movement and camera shaking.

The signal determination unit 38, based on the difference ΔS, determines whether the beacon device 14 and the light generating device 16 are connected.

For example, the signal determination unit 38 may determine that the beacon device 14 and the light generating device 16 are connected in response to the difference ΔS being a threshold value or greater, and determine that that the beacon device 14 and the light generating device 16 are not connected in response to the difference ΔS being smaller than the threshold value. The threshold value is a predetermined value and may be changed by the user such as an operator.

The signal determination unit 38 outputs information indicating the determination result to the controller 30, and the controller 30 causes the display 34 to display the information.

The signal determination unit 38 may determine whether the beacon device 14 and the light generating device 16 are connected based on the threshold value being set in relation to the intensity and S/N ratio of a photoacoustic signal. The signal determination unit 38 may also determine whether the beacon device 14 and the light generating device 16 are connected based on a change of intensity of a photoacoustic signal with time.

FIG. 19 shows the relation among the number of frame integrations, the S/N ratio, and the threshold value. In response to the S/N ratio relative to the number of frame integrations being the threshold value or greater, the signal determination unit 38 determines that the beacon device 14 and the light generating device 16 are connected. Display examples of the determination result will be described below.

FIG. 20 illustrates a screen 80 on the display 34. The screen 80 includes display regions 82, 84, and 86. The display region 82 displays an ultrasonic image, such as a tomographic image and an image based on photoacoustic signals. The display region 84 or the display region 86 displays information indicating the determination result.

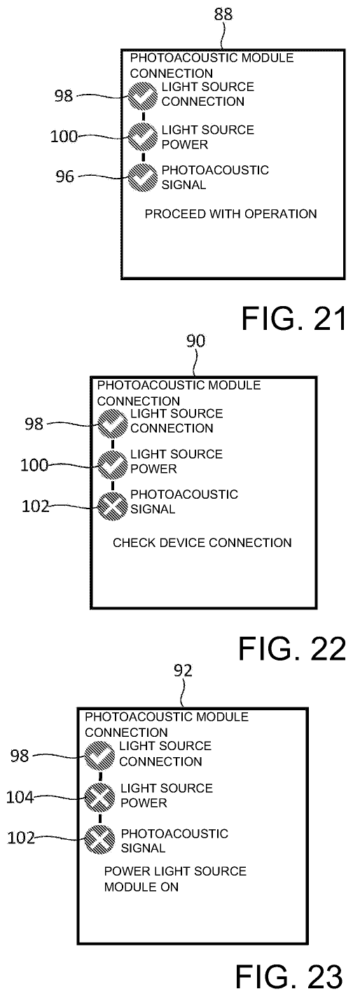

FIG. 21 to FIG. 24 illustrate display examples of determination results.

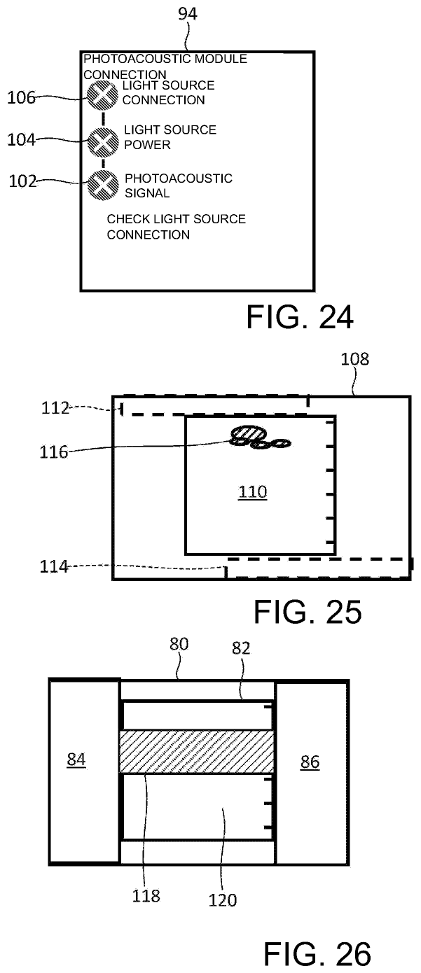

FIG. 21 illustrates an image 88, FIG. 22 illustrates an image 90, FIG. 23 illustrates an image 92, and FIG. 24 illustrates an image 94. Each of the images 88, 90, 92, and 94 represents a determination result.

Each of the images 88, 90, 92, and 94 displays a determination result of “light source connection”, a determination result of “light source ON”, and a determination result of “photoacoustic signal”.

The determination result of “light source connection” refers to a determination result regarding connection of the light source in the light generating device 16. The determination result of “light source power” refers to a determination result as to whether the light source is powered ON or is active in the light generating device 16. The determination regarding “light source connection” and the determination regarding “light source power” may be performed by the light generating device 16 or by the controller 30. The determination result of “photoacoustic signal” refers to a determination result by the signal determination unit 38; that is, a determination result as to whether the beacon device 14 and the light generating device 16 are connected.

The controller 30 displays, in accordance with the determination results, any one of the images 88, 90, 92, and 94 on the display region 84 or the display region 86.

In response to the determination by the signal determination unit 38 that the beacon device 14 and the light generating device 16 are connected, the controller 30 displays the image 88 illustrated in FIG. 21 on the display region 84 or the display region 86. In this example, the light source is connected and active in the light generating device 16. The image 88 includes an image 96, such as a mark or icon, for example, indicating that the beacon device 14 and the light generating device 16 are connected. The image 88 further includes an image 98, such as a mark or icon, for example, indicating that the light source is connected in the light generating device 16, and an image 100, such as a mark or icon, for example, indicating that the light source is active in the light generating device 16. The controller 30 may further display a message “proceed with operation” on the image 88. The controller 30 may show a text sequence, in place of the image 96, indicating that the beacon device 14 and the light generating device 16 are connected, on the image 88.

In response to the determination by the signal determination unit 38 that the beacon device 14 and the light generating device 16 are not connected, the controller 30 displays the image 90 illustrated in FIG. 22 on the display region 84 or the display region 86. The image 90 includes an image 102, such as a mark or icon, for example, indicating that the beacon device 14 and the light generating device 16 are not connected. In this example, the light source is connected and active in the light generating device 16. The image 90, similar to the image 88, includes the images 98 and 100. The controller 30 may display a message “check device connection” on the image 90. The controller 30 may show, on the image 90, a text sequence indicating that the beacon device 14 and the light generating device 16 are not connected, in place of the image 102.

When the light source is powered OFF or inactive, the controller 30 displays the image 92 illustrated in FIG. 23 on the display region 84 or the display region 86. In this case, the image 92 includes an image 104, such as a mark or icon, for example, indicating that the light source is powered OFF or inactive. The controller 30 may further display a message, for example, that encourages the user to check the power source, on the image 92. With the light source being inactive, a photoacoustic signal is not received; therefore, the image 102 is also displayed.

When the light source is not connected in the light generating device 16, the controller 30 displays the image 94 illustrated in FIG. 24 on the display region 84 or the display region 86. In this case, the image 94 includes an image 106, such as a mark or icon, for example, indicating that the light source is not connected. The controller 30 may further display a message, for example, that encourages the user to check the connection of the light source on the image 94. With the light source being not connected, it is determined that light source is powered OFF or inactive and a photoacoustic signal is not received. The images 102 and 104 are therefore displayed.

FIG. 25 illustrates another display example of determination results. For example, the controller 30 causes the display 34 to display a screen 108 including display regions 110, 112, and 114.

The display region 110 displays an ultrasonic image, such as a tomographic image and an image based on photoacoustic signals, for example. The display region 112 or the display region 114 displays information indicating determination results. For example, the display region 110 displays an image 116 based on a photoacoustic signal.

The controller 30 may display a message “when an image based on a photoacoustic signal is displayed, proceed with operation” in the display region 112. When the image 116 is displayed, the user such as an operator may determine that the beacon device 14 and the light generating device 16 are connected and proceed with operation.

The controller 30 may display information indicating the level of a photoacoustic signal or the signal intensity in the display region 114. For example, the controller 30 may display, in the display region 114, information indicating a level corresponding to a received photoacoustic signal, among a plurality of levels previously determined in accordance with the signal intensities. Specifically, in response to a received photoacoustic signal having a high intensity, the controller 30 may display information indicating “photoacoustic signal level: HIGH” in the display region 114.

The signal determination unit 38 may calculate a likelihood of a determination result, and the controller 30 may cause the display 34 to display information indicating the likelihood.

Reception of Photoacoustic Signal from Region Corresponding to Photoacoustic Signal Measurement Instrument

The signal determination unit 38 may determine whether the beacon device 14 and the light generating device 16 are connected based on a photoacoustic signal from a region corresponding to the photoacoustic signal measurement instrument. The region corresponding to the photoacoustic signal measurement instrument refers to a region corresponding to a location of the tip end 14a of the beacon device 14 fixed with the fixing portion. By reference to FIG. 13, FIG. 15, and FIG. 26, specific examples will be described. FIG. 26 illustrates a screen 80.

As illustrated in FIG. 13 and FIG. 15, a side face of the photoacoustic signal measurement instrument 60 according to EXAMPLE 2 includes the through hole 64. The through hole 64 is disposed at a location that is a depth d below the top face of the container 62 or the top face including the acoustic window 66. The tip end 14a of the beacon device 14 inserted through the through hole 64 into the container 62 and is fixed to a location the depth d below the top face of the container or adjacent to the location.

The ultrasonic probe 18 is disposed on the top face of the container 62 with the transmission and reception face of the ultrasonic probe 18 in contact with the acoustic window 66. Therefore, the tip end 14a of the beacon device 14, inserted through the through hole 64 into the container 62 and supported by the container 62, is located at or in the vicinity of a point the distance d apart from the transmission and reception face of the ultrasonic probe 18.

The signal determination unit 38 determines whether the beacon device 14 and the light generating device 16 are connected based on a photoacoustic signal received from the location the distance d apart from the transmission and reception face of the ultrasonic probe 18 or at the location of depth d, or from a region around the location. A region 118 illustrated in FIG. 26 corresponds to the region including the location the distance d apart from the transmission and reception face of the ultrasonic probe 18 or the location of depth d. The region 118 is a region having the location the distance d apart from the transmission and reception face at its center, and having predetermined widths above and under the center, respectively.

The signal determination unit 38 determines whether the beacon device 14 and the light generating device 16 are connected based on a photoacoustic signal received from the region 118. The signal determination unit 38 may determine whether or not the beacon device 14 and the light generating device 16 are connected based on a difference ΔS obtained from photoacoustic signals received from the region 118.

The region 118 includes the tip end 14a of the beacon device 14. It is therefore assumed that a photoacoustic signal received from the region 118 is a photoacoustic signal emitted from the photoacoustic wave source 14d. Use of the photoacoustic signal received from the region 118 therefore enables determination of whether the beacon device 14 and the light generating device 16 are connected, with a signal received from regions not including the tip end 14a being excluded.

The signal determination unit 38 may determine whether the beacon device 14 and the light generating device 16 are connected based on a ratio between a photoacoustic signal from a region corresponding to the photoacoustic signal measurement instrument and a photoacoustic signal received from other regions.

In the example illustrated in FIG. 26, the region 118 is a region corresponding to the photoacoustic signal measurement instrument 60, and specifically a region corresponding to a location where the through hole 64 is disposed or the location at the depth d. A region 120 outside the region 118 corresponds to the region other than the region corresponding to the photoacoustic signal measurement instrument 60. The region 120 thus does not correspond to the location at the depth d.

The region 118 includes the location where the tip end 14a of the beacon device 14 is disposed. It is therefore assumed that a photoacoustic signal received from the region 118 is a photoacoustic signal emitted from the photoacoustic wave source 14d. The region 118 may therefore be determined as an S region where photoacoustic signals are acquired. Meanwhile, the region 120 does not include the location where the tip end 14a of the beacon device 14 is disposed. It is therefore assumed that a photoacoustic signal received from the 120 is a signal such as noise, not a photoacoustic signal emitted from the photoacoustic wave source 14d. The region 120 may therefore be determined as an N region where signals such as noise are acquired.

The signal determination unit 38 determines whether the beacon device 14 and the light generating device 16 are connected based on a ratio of a photoacoustic signal (S signal) acquired from the region 118 or the S region and a photoacoustic signal (N signal) acquired from the region 120 of the N region, which may be regarded as an S/N ratio.

The signal determination unit 38, in response to the above-described ratio being a threshold value or greater, determines that the beacon device 14 and the light generating device 16 are connected, and in response to the above-described ratio being less than the threshold value, determines that the beacon device 14 and the light generating device 16 are not connected.

As described above, the ratio of the photoacoustic signals acquired separately from the region 118 and the region 120 is used. This enables determination of whether the beacon device 14 and the light generating device 16 are connected, with the effect of a signal received from a region not including the tip end 14a being reduced.

1. A photoacoustic signal measurement instrument for use in measurement of a photoacoustic signal from a photoacoustic wave source disposed at a tip end of a device to be inserted into a subject,
the photoacoustic signal measurement instrument comprising:
a holder portion configured to house the tip end of the device, the holder portion holding a substance that allows a photoacoustic signal to pass through; and
a fixing portion configured to fix a relation between a location where an ultrasonic probe is disposed and a location of the tip end of the device, without the ultrasonic probe that receives a photoacoustic signal contacting the tip end of the device.

2. The photoacoustic signal measurement instrument according to claim 1, further comprising:
a container portion configured to house the device, wherein
the holder portion is a recess portion on a surface of the container portion,
the tip end of the device that is housed within the container portion is contained in the recess portion, and
the container portion includes a space outside the recess portion, the space configured to receive the ultrasonic probe facing the recess portion.

3. The photoacoustic signal measurement instrument according to claim 2, wherein
the recess portion has a bottom face including a slope face.

4. The photoacoustic signal measurement instrument according to claim 1, wherein
the holder portion is a container containing the substance,
the fixing portion is a through hole disposed on a surface of the container and communicating with an interior of the container, the through hole configured to allow the tip end of the device that is inserted to extend through, and
the container comprises an acoustic window configured to receive the ultrasonic probe outside the container, the acoustic window allowing a photoacoustic signal from the photoacoustic wave source to pass through.

5. A photoacoustic signal measurement system, comprising:
a photoacoustic signal measurement instrument for use in measurement of a photoacoustic signal from a photoacoustic wave source disposed at a tip end of a device to be inserted into a subject; and
a determination unit, wherein
the photoacoustic signal measurement instrument comprises:
a holder portion configured to house the tip end of the device, the holder portion holding a substance that allows a photoacoustic signal to pass through; and
a fixing portion configured to fix a relation between a location where an ultrasonic probe is disposed and a location of the tip end of the device, without the ultrasonic probe that receives a photoacoustic signal contacting the tip end of the device,
the photoacoustic wave source is irradiated with light from a light generating device via the device to thereby emit a photoacoustic signal, and
the determination unit is configured to determine connection between the light generating device and the device based on a photoacoustic signal emitted from the photoacoustic wave source and received by the ultrasonic probe.

6. The photoacoustic signal measurement system according to claim 5, further comprising:
a controller configured to control emission of light from the light generating device to the photoacoustic wave source and reception of a photoacoustic signal from the photoacoustic wave source, wherein
the controller is configured to control execution of first processing including emission of light and reception of a photoacoustic signal in response to the emission, and second processing including reception of a photoacoustic signal with no light being emitted.

7. The photoacoustic signal measurement system according to claim 6, further comprising:
a signal processor configured to process a photoacoustic signal received by the ultrasonic probe, wherein
the signal processor is configured to calculate a difference between a photoacoustic signal that is received by execution of the first processing and a photoacoustic signal that is received by execution of the second processing, and
the determination unit is configured to determine a connection state between the light generating device and the device based on the difference.

8. The photoacoustic signal measurement system according to claim 5, wherein
the determination unit is configured to determine a connection state between the light generating device and the device based on a photoacoustic signal from a region corresponding to the photoacoustic signal measurement instrument.

9. The photoacoustic signal measurement system according to claim 8, wherein
the region corresponding to the photoacoustic signal measurement instrument is a region corresponding to a location of the tip end of the device fixed by the fixing portion.

10. The photoacoustic signal measurement system according to claim 9, wherein
the determination unit is configured to determine a connection state between the light generating device and the device based on a ratio of a photoacoustic signal from the region corresponding to the photoacoustic signal measurement instrument and a photoacoustic signal from other regions.
